(19) United States
(12) Patent Application Publication
(10) Pub. No.: US 2021/0031176 A1
(43) Pub. Date: Feb. 4, 2021

(54) CATALYSTS, SYSTEMS, AND PROCESSES FOR REGULATING A CONTACTING STATE IN PRODUCING LIGHT OLEFINS FROM PARAFFINS

(71) Applicant: SMH Co., Ltd, Bangkok (TH)

(72) Inventors: Kongkiat SURIYE, Samutprakan (TH); Wuttithep JAREEWATCHARA, Bangkok (TH); Atsadawuth SIANGSAI, Bangkok (TH)

(21) Appl. No.: 16/967,356

(22) PCT Filed: Feb. 4, 2019
(86) PCT No.: PCT/IB19/50881

§ 371 Date: Aug. 4, 2020

Related U.S. Application Data
(60) Provisional application No. 62/626,219, filed on Feb. 5, 2018.

(51) Int. Cl. B01J 35/00; B01J 23/62; B01J 23/02; B01J 29/16; B01J 23/30; B01J 21/08; C07C 5/48; C07C 6/04

(52) U.S. Cl. CPC B01J 35/0006; C07C 2521/08; B01J 23/62; B01J 23/02; B01J 29/166; B01J 23/30; B01J 21/08; C07C 5/48; C07C 6/04; C07C 2523/62; C07C 2521/10; C07C 2521/04; C07C 2529/16; C07C 2523/30; B01J 23/626

(57) ABSTRACT

The present invention relates to catalysts, catalyst systems, and processes for the production of valuable light olefins, such as ethylene, from paraffinic hydrocarbons, such as propane, through dehydrogenation and metathesis. The contacting state between dehydrogenation and metathesis catalysts can advantageously be manipulated using an inert or relatively inert coating or outer shell that provides a degree of physical separation between catalytically active centers or inner cores. This has been discovered to significantly increase olefin selectivity (i.e., reduce undesired hydrogenation/hydrogenolysis side reactions) without an appreciable paraffin conversion deficit, such that the overall yield of desired olefinic hydrocarbons such as ethylene is thereby significantly increased.

### CROSS-REFERENCE TO RELATED APPLICATIONS

This application claims priority to U.S. provisional application No. 62/626,219, filed Feb. 5, 2018, the content of which is hereby incorporated by reference in its entirety.

### FIELD OF THE INVENTION

The invention relates to coated dehydrogenation catalysts, coated olefin metathesis catalysts, catalyst systems comprising one or both of these catalysts, and dehydrogenation and olefin metathesis processes for converting a paraffinic hydrocarbon (e.g., propane) to at least two olefinic hydrocarbons (e.g., ethylene and butene) having different carbon numbers, relative to the paraffinic hydrocarbon.

### DESCRIPTION OF RELATED ART

The global ethylene demand is currently high and expected to increase, in view of anticipated growth in the major end product polyethylene, used in plastic bags, films, bottles, etc. across a wide range of industries. Intermediate products made from ethylene include ethylene oxide and ethylene glycol for the production of polyethylene terephthalate (PET) resins for PET fiber, as well as ethylene dichloride for the production of polyvinylchloride (PVC) plastic used in construction and piping. The increasing applications of these and other ethylene-based intermediates are expected to further increase the demand for ethylene. For example, the production of textile fibers from ethylene oxide is growing significantly, especially in Asia-Pacific. Also, producers of ethylene oxide have been able to profit from the worldwide growing substitution of glass by PET bottles and containers. Overall, the global ethylene market is regarded as being segmented into high density polyethylene (HDPE), low-density polyethylene (LDPE) and linear low density polyethylene (LLDPE) end products, as well as the above-noted intermediates, mainly used for the production of plastics.

Currently, the major route for ethylene production is steam cracking of paraffinic hydrocarbons, the main sources of which are obtained from crude oil refining, particularly light gas oil, including liquefied petroleum gas (LPG), and naphtha. Steam cracking, however, involves a very complex combination of reaction and gas recovery systems. Feedstock is charged to a thermal cracking zone in the presence of steam at effective conditions to produce a pyrolysis reactor effluent gas mixture. The mixture is then stabilized and separated into purified components through a sequence of cryogenic and conventional fractionation steps. Generally, the product ethylene is recovered as a low boiling fraction, such as an overhead stream, from an ethylene/ethane splitter column requiring a large number of theoretical stages due to the similar relative volatilities of the ethylene and ethane being separated. The cracking of olefins in hydrocarbon feedstocks, to produce these lighter olefins from C₄ mixtures obtained in refineries and steam cracking units, is described in U.S. Pat. Nos. 6,858,133; 7,087,155; and 7,375,257. Other significant sources of ethylene include byproducts of fluid catalytic cracking (FCC) and resid fluid catalytic cracking (RFCC), normally targeting gasoline production. FCC is described, for example, in U.S. Pat. No. 4,288,688 and elsewhere.

Yields of ethylene and other light olefins from steam cracking and other processes may be improved using known methods for the metathesis or disproportionation of C₄ and heavier olefins. Metathesis may also be combined with a separate cracking step in the presence of a zeolitic catalyst, as described, for example, in U.S. Pat. Nos. 5,026,935 and 5,026,936. Light olefins, namely propylene and butene that can be used for ethylene production through metathesis and/or cracking, can also be produced through a dedicated process of paraffin dehydrogenation, as described in U.S. Pat. No. 3,978,150 and elsewhere. However, the significant capital cost of a propane dehydrogenation plant is normally justified only in cases of large-scale propylene production units (e.g., typically 250,000 metric tons per year or more). The substantial supply of propane feedstock required to maintain this capacity is typically available from propane-rich liquefied petroleum gas (LPG) streams from gas plant sources. The production of light olefins directly from paraffins through a combination of dehydrogenation and disproportionation reaction steps is described in U.S. Pat. No. 3,445,541. Other processes for the targeted production of light olefins involve high severity catalytic cracking of naphtha and other hydrocarbon fractions. A catalytic naphtha cracking process of commercial importance is described in U.S. Pat. No. 6,867,341.

The desire for light olefins from alternative, non-petroleum based feeds has also led to the use of oxygenates such as alcohols and, more particularly, methanol, ethanol, and higher alcohols or their derivatives. Methanol, in particular, is useful in a methanol-to-olefin (MTO) conversion process described, for example, in U.S. Pat. No. 5,914,433. The yield of light olefins from such processes may be improved using olefin cracking to convert some or all of the C₄⁺ product of MTO in an olefin cracking reactor, as described in U.S. Pat. No. 7,268,265. An oxygenate to light olefins conversion process in which the yields of olefin products of varying carbon numbers may be adjusted through the use of dimerization and metathesis is described in U.S. Pat. No. 7,586,018.

Despite the use of various dedicated and non-dedicated routes for generating ethylene and other light olefins industrially, there remains an ongoing need for olefin production processes with reduced complexity, improved economics, and/or superior yields relative to current processes.

### SUMMARY OF THE INVENTION

The present invention relates to catalysts, catalyst systems, and processes for the production of valuable light olefins, such as ethylene, from paraffinic hydrocarbons, such as propane, through dehydrogenation and metathesis. Advantageously, a single catalyst system can be used for both of these reaction steps. In the case of a feed comprising propane, for example, its dehydrogenation to propylene and hydrogen proceeds according to:

C₃H→C₃H₆+H₂ (reaction 1—dehydrogenation).

The resulting propylene can then undergo metathesis or conversion to olefin products of lower and higher carbon numbers, in this case ethylene and butene, respectively. More particularly, olefin metathesis results in redistribution of alkylidene radicals that would be generated upon cleavage of the carbon-carbon double bond of an acyclic olefin. The self-metathesis of the asymmetrical olefin propylene, therefore, results in rearrangement of the olefinic carbon atom substituents to produce both ethylene and butene according to the following reaction:

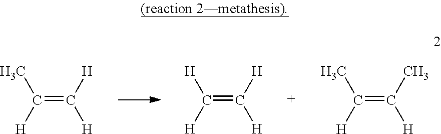

Aspects of the invention are associated with important discoveries regarding the above reactions proceeding in combination (e.g., sequentially) to produce, from a feed comprising one or more paraffinic hydrocarbons, one or more light olefins selected from the group consisting of C₂-C₅ monoolefins and combinations thereof. Representative paraffinic hydrocarbons include C₂-C₆ paraffins, such as ethane, propane, butane, pentane, and hexane, including all structural isomers of the latter three. C₂-C₅ monoolefins include ethylene, propylene, butene (including any of its positional and structural isomers, namely butene-1, cis-butene-2, trans-butene-2, and isobutylene), and pentene (including any of its positional and structural isomers). Particular embodiments are directed to the production of ethylene from propane.

Without being bound by theory, experimental studies provide evidence that the contacting state between catalysts for these reaction steps can significantly impact the overall conversion and selectivity profile of a given catalyst system. It has been found, for example, that a “high” contacting state between dehydrogenation and metathesis catalysts causes a relatively fast consumption of the dehydrogenation product (e.g., propylene), resulting in high overall conversion of the paraffinic hydrocarbon (e.g., propane). That is, rapid depletion of the dehydrogenation product shifts the equilibrium-limited dehydrogenation reaction in the forward direction. Alternatively, a “low” contacting state between the catalysts has the opposite effect on conversion. However, desirable changes in conversion (e.g., propane conversion) due to the catalyst contacting state are met with undesirable changes in reaction selectivity to olefinic hydrocarbon(s) (e.g., selectivity to ethylene and/or butene), including any of those produced by the metathesis reaction described above. Such losses in selectivity, i.e., the percentage of the converted paraffinic hydrocarbon(s) that manifest in olefinic hydrocarbon(s), result from undesired side reactions such as re-hydrogenation of the dehydrogenation product to the starting paraffinic hydrocarbon and/or hydrogenolysis to produce low molecular weight paraffinic hydrocarbons (e.g., methane).

Advantageously, aspects of the invention relate to the ability to manipulate a dehydrogenation catalyst/metathesis catalyst contacting state, thereby influencing the reaction conversion and selectivity profile to achieve an overall yield of olefinic hydrocarbon(s) beyond that of conventional processes. Those skilled in the art will appreciate that even small increases in product yield can have a profound impact on the economic viability and/or attractiveness of hydrocarbon conversion processes, especially considering their typical scale of commercial operation. In this regard, the surprising impact of the contacting state can lead to significant yield increases of one or more desirable olefinic hydrocarbons such as ethylene, having a high demand and associated market value.

For purposes of explanation, a “low” contacting state and its corresponding, relatively low conversion of the paraffinic hydrocarbon(s) with relatively high selectivity to olefinic hydrocarbon(s), can be achieved using separated dehydrogenation and metathesis catalysts, such as being disposed in separate dehydrogenation and metathesis reactors configured in series, with the effluent of the dehydrogenation reactor being passed to the inlet of the metathesis reactor. A higher contacting state results from disposing the dehydrogenation and metathesis catalysts in a stacked bed relationship, but divided using a layer of inert material along the direction of the feed and product flow (i.e., between the outlet of the dehydrogenation catalyst bed and inlet of the metathesis catalyst bed). A still higher contacting state is attained if this inert dividing material is removed, such that the beds of different catalysts are stacked directly adjacent one another. Yet a higher contacting state results from a single bed of a physical, uniform mixture of the two catalysts. Along this spectrum of contacting states from lowest to highest, conversion directionally increases and selectivity decreases, as described above, assuming all other operating parameters are maintained the same.

The contacting state can also be varied, however, using an inert or relatively inert (i.e., relative to the base catalytic activity of the dehydrogenation or metathesis catalyst) coating or outer shell that provides a degree of physical separation of a more catalytically active “inner core” of the catalyst. Surprisingly, it has now been discovered that this strategy for manipulating the contacting state can significantly increase olefin selectivity (i.e., reduce undesired hydrogenation/hydrogenolysis side reactions) without the appreciable paraffin conversion deficit obtained when the contacting state is increased according to other methods. The overall yield of desired olefinic hydrocarbons such as ethylene is thereby significantly increased.

Representative processes therefore comprise contacting a feed comprising one or more paraffinic hydrocarbons such as propane with a dehydrogenation and metathesis catalyst system in which at least a portion of one or both of the dehydrogenation catalyst and the metathesis catalyst is coated with an outer shell that results in an advantageous contacting state, and the associated tradeoff between conversion and selectivity, as described above. Consequently, a desired olefinic hydrocarbon such as ethylene can be produced with a high yield. According to particular embodiments, a paraffinic hydrocarbon (e.g., propane) is present in the feed at a predominant concentration (e.g., greater than about 50% by volume and/or greater than that of any paraffinic hydrocarbon having a different carbon number, such as butane), and the product resulting from the conversion of this paraffinic hydrocarbon comprises at least two olefinic hydrocarbons (e.g., ethylene and butene) having different carbon numbers relative to the paraffinic hydrocarbon that is present in the feed at the predominant concentration. According to preferred embodiments, as a result of the metathesis, the selectivity to these olefinic hydrocarbons, or possibly even to only one of these olefinic hydrocarbons of particular interest/value (e.g., ethylene), is at least about 15% (i.e., 15% of the converted moles of paraffinic hydrocarbon(s) manifest as moles of the olefinic hydrocarbons). Also, according to particular embodiments, this selectivity to at least one olefinic hydrocarbon may be achieved at a conversion of the paraffinic hydrocarbon, which is present in the feed at a predominant concentration as described above, of at least about 40%.

These and other aspects and embodiments associated with the present invention are apparent from the following Detailed Description.

### BRIEF DESCRIPTION OF THE DRAWINGS

FIG. 1A is a transmission electron microscopy (TEM) image of a metathesis catalyst particle, having a composition and made by a procedure as described herein.

FIG. 1B is a TEM image of a metathesis catalyst particle as shown in FIG. 1A, but having a coating of mesoporous silica (mSiO₂).

FIG. 2 is a TEM image that shows structural features of a coated metathesis catalyst particle as shown in FIG. 1B.

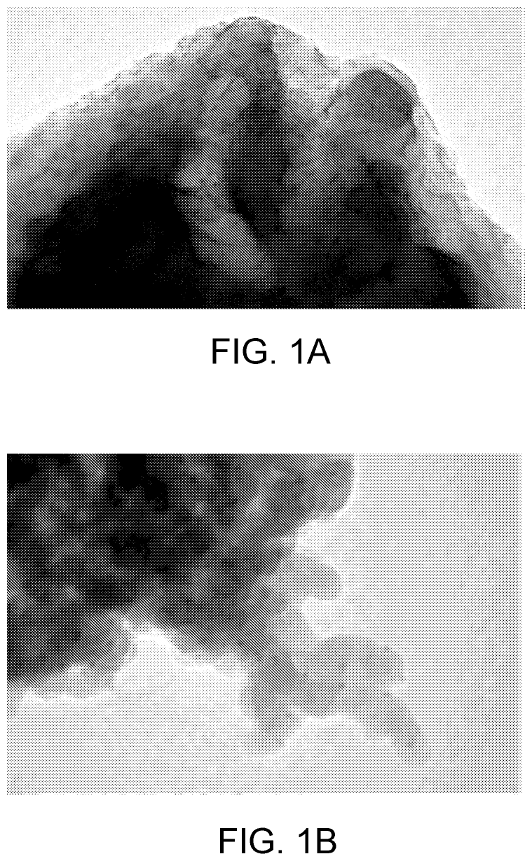

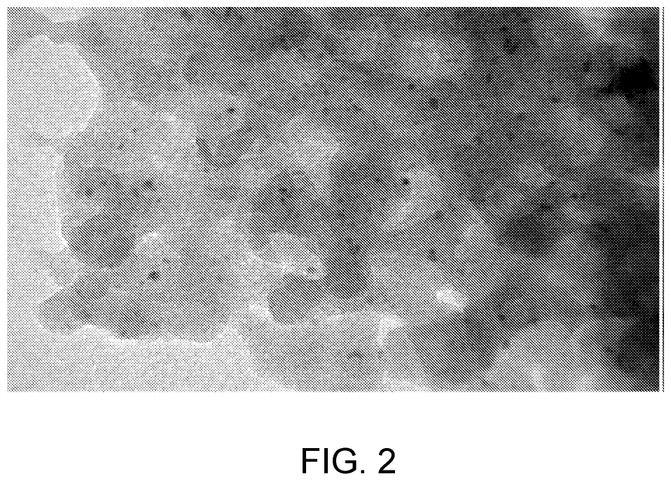

The full width of the images of FIGS. 1A, 1B, and 2 provides a view spanning approximately 60 nanometers (nm).

### DETAILED DESCRIPTION

As discussed above, the present invention relates to embodiments including (i) coated dehydrogenation catalysts for performing dehydrogenation reactions such as reaction 1 above, (ii) coated metathesis catalysts for performing olefin metathesis reactions such as reaction 2 above, (iii) catalyst systems comprising dehydrogenation catalyst particles and olefin metathesis catalyst particles (e.g., as a uniform mixture), in which at least a portion of one or both of these types of catalyst particles are particles of a dehydrogenation catalyst or an olefin metathesis catalyst as described herein (e.g., according to embodiments (i) or (ii) above), and (iv) dehydrogenation and metathesis processes for performing a dehydrogenation reaction such as reaction 1 above in combination with (e.g., preceding) olefin metathesis reaction such as reaction 2 above, by contacting a feed comprising a paraffinic hydrocarbon with a catalyst system as described herein (e.g., according to embodiment (iii) above). Such catalysts, catalyst systems, and processes are associated with unique advantages, particularly in terms of providing a high yield of olefinic hydrocarbons such as ethylene.

As used herein, generic terms for hydrocarbons having a particular carbon number are meant to encompass all double bond positional isomers and/or structural isomers (where applicable). For example, the terms “butene,” “butenes,” and “butene isomers” are meant to encompass butene-1, cis-butene-2. trans-butene-2, and isobutylene. The term “carbon number,” in reference to olefinic or paraffinic hydrocarbons, for example in phrases such as “same carbon number” or “different carbon number,” is meant to refer to the number of carbon atoms present in that olefinic or paraffinic hydrocarbon.

For element group designations described herein, reference is made to the “CRC Handbook of Chemistry and Physics”, 76^{th} Edition (1995-1996), by David R. Lide, published by CRC Press, Inc. (USA), in which the groups of the periodic table are numbered as Groups 1 to 18.

As used herein, the term “mixed metal oxide” as used, for example, in the phrases “dehydrogenation support mixed metal oxide” and “metathesis support mixed metal oxide” refers to a mixture of metal oxides, such as a mixture of magnesium oxide (MgO) and aluminum oxide (Al₂O₃). The term “mixed metal oxide” extends to layered double hydroxides, which are particular materials from which mixtures of metal oxides, corresponding to the hydroxides of the layered double hydroxide, can be obtained by thermal treatment. A representative thermal treatment comprises heating the layered double hydroxide, in this case serving as a “precursor” of the mixed metal oxide, for an extended time (e.g., from about 1 to about 15 hours, such as from about 1 to about 10 hours or from about 5 to about 10 hours) and at an elevated temperature (e.g., from about 300° C. to about 800° C. such as from about 500° C. to about 750° C.) to transform the precursor layered double hydroxide to a mixture of metal oxides (i.e., the mixed metal oxide), which may or may not retain the layered structure of the precursor. Therefore, a “mixed metal oxide,” according to this disclosure, may either be present as (i.e., in the form of), or derived from, a layered double hydroxide (LDH). Layered double hydroxides and their preparation are described, for example, in WO 2016/120423. These are also known as anionic clays or hydrotalcite-like materials, having a unique structure with positively charged layers and charge-balancing anions and water interlayers. A general chemical formula of an LDH is:

[M^{y+}₁₋ₓM′^{z+}ₓ(OH)₂]^{a+}(A^{r-})ₙ.b(H₂O) (I).

In formula (I) above, M and M′ are first and second metals that may be independently selected from alkali metals, alkaline earth metals, transition metals, and other metals. In representative embodiments, the first metal, M, may be selected from the group consisting of Li. Mg, Zn, Fe, Ca, Ni, Co, Mn, and Cu, with Ca and Mg being preferred. In other representative embodiments, the second metal, M′, may be selected from the group consisting of Al, Ga, Y. In, Fe, Co, Ni, Mn, Cr, Ti, V, Zr, and La, with Al being preferred. In formula (I) above, A is an anion, examples of which include fluoride, chloride, bromide, iodide, carbonate, bicarbonate, hydrogen phosphate, dihydrogen phosphate, nitrate, nitrite, borate, sulfate, phosphate, and hydroxide, with carbonate and nitrate being preferred. In formula (I) above, the value of x is preferably from 0.1 to 0.9; the value of y, representing the charge of the first metal, M, is preferably 1 or 2; the value of z, representing the charge of the second metal, M′, is preferably 3 or 4; the value of a is (1−x)·y+x·z−2; the value of r, representing the charge of the anion, is preferably 1, 2, or 3 (giving charges of “r−” that are namely −1, −2, or −3, respectively); the value of n is a/r; and the value of b, representing the relative number of water molecules, is preferably from 0 to 10.

In some cases, ranges of ratios of two components (i):(ii), ranges of ratios of three components (i):(ii):(iii), etc. are expressed using ranges of values for the individual components to which these ratios apply. For example, in the case of a range of weight ratios (i):(ii) expressed as “1:1-3.” the second component value may range from 1 to 3 parts by weight, relative to the first component, such that this range of weight ratios encompasses any weight ratio of (i):(ii) from 1:1 to 1:3. Equivalently, in the case of weight ratios (i):(ii):(iii) expressed as “0.5-2:0-2:0.5-2,” the first and third components may range from 0.5 to 2 parts by weight, relative to the second component, and relative to each other, and the second component may range from 0-2 parts by weight, relative to the first and third components. Therefore, in the particular case of the second component being 0, this range of weight ratios encompasses any weight ratio of (i):(iii) from 1:4 (or 0.5:2) to 4:1 (or 2:0.5).

Aspects of the invention relate to advantages, as described herein, associated with manipulating a contacting state between dehydrogenation and metathesis catalysts, by providing an outer shell or coating that is disposed peripherally (e.g., externally) about an inner core body comprising a dehydrogenation active catalytic component or an olefin metathesis active catalytic component. This outer shell or coating may comprise a buffering metal oxide that acts primarily to provide a desired degree of physical separation (and lack of direct contacting) between the different catalyst types, for example when packed in a uniform mixture, without necessarily providing substantial catalytic activity itself. In some embodiments, a dehydrogenation outer shell comprises a dehydrogenation buffering metal oxide and substantially lacks a dehydrogenation active catalytic component, such as that component present in a dehydrogenation inner core body, and/or otherwise substantially lacks the dehydrogenation catalytic activity of the dehydrogenation inner core body. Likewise, in some embodiments, a metathesis outer shell comprises a metathesis buffering metal oxide and substantially lacks an olefin metathesis active catalytic component, such as that component present in a metathesis inner core body, and/or otherwise substantially lacks the olefin metathesis catalytic activity of the metathesis inner core body.

Regardless of the particular type of coating or outer shell that is disposed peripherally about an inner core body having dehydrogenation activity (dehydrogenation inner core body) or disposed peripherally about an inner core body having metathesis activity (metathesis inner core body), it can be appreciated, in view of the present disclosure, that the desired, physical separation between catalytically active inner core bodies may be achieved even if other coatings or shells are present. Accordingly, a catalyst comprising such inner core body and also comprising an outer shell disposed peripherally about such inner core body encompasses catalysts having one or more additional material layers (i) external to the outer shell, and/or (ii) between the outer shell and the inner core body. Accordingly, the designation of “outer shell” refers to its position relative to the “inner core body,” but does not necessarily mean that the “outer shell” forms the outermost layer or includes the external surface of a catalyst particle. Likewise, the designation of “inner core body” does not necessarily require that this be the innermost (central) body of a catalyst particle, such that “inner core body” encompasses catalysts having additional material within (central to) this inner core body. According to preferred embodiments, (i) the outer shell forms an outermost layer and includes the external surface of a catalyst particle, (ii) the outer shell is disposed directly on the inner core body (without any intervening layer), (iii) the inner core body is the innermost (central) body of a catalyst particle, or (iv) any one or more of (i), (ii) and (iii) in combination.

Application of the outer shell to the inner core body may be performed, for example, by preparing a sol-gel of the material to be formed into the outer shell (e.g., a sol-gel of a buffering metal oxide such as magnesium oxide or silicon oxide, which may be present more particularly in the form of mesoporous silica). The inner core body may then be immersed in such sol-gel, removed from the sol-gel with a coating of the so-gel thereon, and subjected to a drying step to evaporate the solvent and form the outer shell. Alternatively, the sol-gel may be applied to the inner core body by spin coating, optionally using electrical ion control, or otherwise by spray coating. According to another embodiment, a fluidized bed coating process may be used. In one embodiment of particular interest, a sol-gel of the material to be formed into the outer shell may be applied (coated) onto the inner core body while in the form of a fluidized bed of particles. For example, particles of the inner core body may be fluidized and contacted with the sol-gel, such as in the case of this component being provided to the fluidized bed as a spraying liquid or spraying gel. That is, the sol-gel may be sprayed as fine droplets or spray particles, such as droplets or particles having been atomized (e.g., using an atomizing gas such as atomizing air), onto the particles of the inner core body while being fluidized. Generally, fluidized bed coating of the inner core body, by the application (e.g., by spraying) of the material to be formed into the outer shell provides a desired, uniformly coated dehydrogenation catalyst or metathesis catalyst. According to particular embodiments, the buffering metal oxide of either a dehydrogenation outer shell or a metathesis outer shell may be independently selected from magnesium oxide, calcium oxide, silicon oxide (silica), strontium oxide, and other metal oxide(s) preferably having low acidity and low cracking activity. The dehydrogenation outer shell may also comprise a dehydrogenation buffering mixed metal oxide of the dehydrogenation buffering metal oxide and a further dehydrogenation outer shell metal oxide. Likewise, the metathesis outer shell may also comprise a metathesis buffering mixed metal oxide of the metathesis buffering metal oxide and a further metathesis outer shell metal oxide. Any of these buffering metal oxides or mixed metal oxides, described herein, singly or in combination, may be present in the dehydrogenation outer shell or metathesis outer shell in an amount of generally at least about 90% by weight, typically at least about 95% by weight, and often at least about 99% by weight, of the outer shell.

The use of an outer shell disposed peripherally about either a dehydrogenation inner core body or a metathesis inner core body results in a coated dehydrogenation catalyst or coated metathesis catalyst, respectively. In such coated catalysts, the inner core body may have any suitable shape, such as spherical (e.g., when prepared by oil dropping) or cylindrical (e.g., when prepared by extrusion). In general, coating does not alter the overall shape or form of the inner core body, in providing the resulting coated catalyst. Other shapes of the inner core body and resulting, coated catalyst include spheroidal, hemispherical, hemispheroidal, or cubic. The inner core body and resulting coated catalyst may otherwise be in the form of a ring, a tablet, or a disc. The outer shell may be present in an amount for providing an advantageous contacting state or separation between inner core bodies having catalytic activity, thereby balancing conversion and selectivity as described above. For example, a dehydrogenation outer shell may be present in a coated dehydrogenation catalyst, or a metathesis outer shell may be present in a coated metathesis catalyst, in an amount generally from about 10% to about 85% by weight, typically from about 20% to about 75% by weight, and often from about 35% to about 60% by weight, of the respective catalyst. In particular embodiments in which the inner core body of a coated dehydrogenation catalyst or a coated metathesis catalyst is spherical, the ratio of the average thickness of the outer shell to the diameter of the inner core body may be from about 1:10 to about 1:1. A coated dehydrogenation catalyst or coated metathesis catalyst, if spherical, may have a diameter suitable for use in a fixed bed, for example, in the range generally from about 1 mm to about 10 mm, typically from about 1 mm to about 5 mm, and often from about 1 mm to about 3 mm. Catalyst particles of such coated catalysts having other geometries, and also suitable for use in a fixed bed, include cylindrical catalyst particles (e.g., when prepared by extrusion). If cylindrical, a coated dehydrogenation catalyst or coated metathesis catalyst may have a diameter within any of the ranges for diameter given above, with respect to spherical catalysts. For example, extrudates may be formed having diameters of 1.59 mm ( 1/16 inch), 3.18 mm (⅛ inch), or 6.35 mm (¼ inch). Cylindrical catalysts may also have a length generally from about 1 mm to about 10 mm, typically from about 1 mm to about 5 mm, and often from about 1 mm to about 3 mm Alternatively, such coated catalysts may have particle sizes suitable for fluidized bed operation, for example in the range generally from about 10 μm to 500 μm and typically from about 50 μm to about 300 μm.

Coated Dehydrogenation Catalysts

Representative coated dehydrogenation catalysts therefore comprise a dehydrogenation inner core body and a dehydrogenation outer shell disposed peripherally with respect to this inner core body, such as over all or at least a portion of its outer surface. The outer shell may comprise a dehydrogenation buffering metal oxide and may substantially lack a dehydrogenation active catalytic component that is present in the inner core body. In addition to this dehydrogenation active catalytic component, the inner core body may comprise a dehydrogenation support metal oxide, optionally with other support constituents (described below), which acts as a carrier of this component, for example with this component being dispersed uniformly or possibly non-uniformly (e.g., preferentially near the outer surface) within the dehydrogenation support metal oxide and optional other support constituents. The dehydrogenation support metal oxide and the dehydrogenation buffering metal oxide may independently be magnesium oxide, calcium oxide, silicon oxide (silica), strontium oxide, or other metal oxide having low acidity and therefore low cracking activity. The dehydrogenation support metal oxide may also be aluminum oxide. In some embodiments, the dehydrogenation support metal oxide and the dehydrogenation buffering metal oxide may be the same type of metal oxide (e.g., magnesium oxide). In more particular embodiments the primary, or possibly only, difference in composition between the dehydrogenation inner core body and dehydrogenation outer shell may be the presence of the dehydrogenation active catalytic component in the former and lack, or substantial lack, of this component in the latter.

According to particular embodiments relating to coated dehydrogenation catalysts, the dehydrogenation support metal oxide may be present in a mixed metal oxide, together with at least one further dehydrogenation support metal oxide (as a support constituent), at various weight ratios. For example, a representative dehydrogenation support mixed metal oxide may be an oxide of a first metal selected from the group consisting of Li, Mg, Zn, Fe, Ca, Ni, Co, Mn, and Cu, and the further dehydrogenation support metal oxide may be an oxide of a second metal selected from the group consisting of Al, Ga, Y, In, Fe, Co, Ni, Mn, Cr, Ti, V, Zr, and La. In a specific embodiment, the dehydrogenation inner core body may comprise a dehydrogenation support mixed metal oxide of magnesium oxide (as the support metal oxide) and aluminum oxide (as the further dehydrogenation support metal oxide). These may be present in the dehydrogenation inner core body in a magnesium oxide:aluminum oxide weight ratio from about 1:1 to about 5:1 or from about 1:1 to about 3:1, such as about 3:1. A dehydrogenation support mixed metal oxide may be present as, or derived from, a layered double hydroxide (LDH) as described above, such as a magnesium-aluminum LDH, in which the first and second metals M and M′, of the general LDH formula (I) above are Mg and Al, respectively. Alternatively, or in combination (e.g., as a further support constituent), the dehydrogenation inner core body may comprise aluminum oxide (alumina), silicon oxide (silica) or a mixture thereof. According to a specific embodiment, the dehydrogenation inner core body may comprise (i) a dehydrogenation support mixed metal oxide present as, or derived from, a layered double hydroxide (LDH) and/or (ii) aluminum oxide (alumina), silicon oxide (silica) or a mixture thereof, onto which a dehydrogenation active catalytic component, as described herein, is dispersed. For example, the dehydrogenation inner core body may be prepared by physically mixing (i) and/or (ii), preferably wherein (i) is a dehydrogenation support mixed metal oxide that is derived from an LDH. Whether or not the dehydrogenation support metal oxide is present in, or comprises, a mixed metal oxide, this support metal oxide is preferably calcined, meaning that it has been subjected to high temperature (e.g., from 200° C. to 800° C.) to improve dispersion of the dehydrogenation active catalytic component, change structural characteristics of this support metal oxide, and/or volatilize undesired impurities. Calcination may involve subjecting the dehydrogenation support metal oxide to an oxidizing heat treatment, for example under a stream of dry air, at a temperature below the sintering temperature of the support (such as more preferably from about 500° C. to about 650° C.), and for a duration sufficient to eliminate carbon dioxide, for example from 0.1 to 48 hours. The calcination may be conducted at atmospheric pressure or otherwise under elevated pressure or subatmospheric pressure.

In addition to a dehydrogenation support metal oxide, the dehydrogenation inner core body may also comprise a dehydrogenation active catalytic component, such as one or more dehydrogenation active metals (metallic elements having activity for catalyzing dehydrogenation reactions such as reaction 1 above) dispersed on the dehydrogenation support metal oxide as described herein. For example, according to an evaporative impregnation method, a precursor compound of a dehydrogenation active metal (e.g., chloroplatinic acid) is dissolved in solution and contacted with the dehydrogenation support metal oxide. The dehydrogenation active metal, in this case platinum, becomes deposited on the support after evaporation of the impregnation solution. In addition to platinum, representative dehydrogenation active metals may include any one or more of chromium, gallium, potassium, lanthanum, yttrium, ytterbium, and rhenium. One or more of these dehydrogenation active metals may be present in the dehydrogenation inner core body, such as dispersed uniformly or non-uniformly therein, in various oxidation states, including their zero valence or elemental metal form. A given dehydrogenation active metal may alternatively be present at other oxidation states, such as its oxide state (e.g., as chromium oxide), or otherwise in more than a single oxidation state (e.g., in a mixed oxidation state). The one or more dehydrogenation active metals, or compounds (e.g., oxides) of such metals, may be present in a combined amount generally from about 0.05% to about 10%, typically from about 0.1% to about 1%, and often from about 0.1% to about 0.5%, by weight of the dehydrogenation inner core body. The dehydrogenation inner core body may further comprise one or more promoter metals, for promoting the dehydrogenation catalytic activity, which may be deposited on the dehydrogenation support metal oxide as described above, for example together with the dehydrogenation active metal or before or after the deposition of this metal. Representative promoter metals include those selected from Group 13 or Group 14 of the Periodic Table, with gallium, tin, indium and combinations thereof being exemplary. One or more of such promoter metals, or compounds (e.g., oxides) of such metals, may be present in a combined amount generally from about 0.1% to about 10%, typically from about 0.3% to 5%, and often from about 0.3% to about 1%, by weight of the dehydrogenation inner core body. The dehydrogenation support metal oxides or mixed metal oxides, dehydrogenation active catalytic component, and promoter metal(s) may be present in a combined amount of generally at least about 90% by weight, typically at least about 95% by weight, and often at least about 99% by weight, of the inner core body. According to particular embodiments, the inner core comprises platinum as the dehydrogenation active catalytic component (e.g., present in an amount within any of the ranges described above for the one or more dehydrogenation active metals) and further comprises tin and/or indium as the promoter metal (e.g., present in an amount, or combined amount, within any of the ranges described above for the one or more promoter metals).

Particular examples of dehydrogenation inner core bodies comprise (i) chromium oxide as a dehydrogenation active metal (e.g., optionally in combination with any one or more of gallium, tin, or indium as a promoter metal) and aluminum oxide as a dehydrogenation support metal oxide, (ii) chromium oxide as a dehydrogenation active metal (e.g., optionally in combination with any one or more of gallium, tin, or indium as a promoter metal) and silicon oxide as a dehydrogenation support metal oxide, (iii) chromium oxide (e.g., optionally in combination with any one or more of gallium, tin, or indium as a promoter metal) as a dehydrogenation active metal and magnesium oxide as a dehydrogenation support metal oxide, (iv) platinum as a dehydrogenation active metal (e.g., optionally in combination with any one or more of gallium, tin, or indium as promoter metal(s)) and aluminum oxide as a dehydrogenation support metal oxide, (v) platinum as a dehydrogenation active metal (e.g., optionally in combination with gallium, tin, or indium as promoter metal(s)) and silicon oxide as a dehydrogenation support metal oxide, and (vi) platinum as a dehydrogenation active metal (e.g., optionally in combination with gallium, tin, or indium as promoter metal(s)) and magnesium oxide as a dehydrogenation support metal oxide. For any of these exemplary inner core bodies, the support metal oxide may be present in a mixed metal oxide. For example, in the case of (vi) above, the inner core body may more particularly comprise platinum and tin, and also the magnesium oxide may be present in a mixed metal oxide, together with a further dehydrogenation support metal oxide such as aluminum oxide. That is, the inner core may comprise platinum and tin, and may further comprise a dehydrogenation support mixed metal oxide, comprising, for example, magnesium oxide and aluminum oxide, wherein the mixed metal oxide may be derived from an LDH, such as a magnesium-aluminum LDH. In specific embodiments, a dehydrogenation inner core body may comprise, for example, platinum and tin at 0.3% and 1.6% by weight, respectively; platinum and tin at 0.3% and 0.6%, respectively; or platinum and indium at 0.3% and 0.6%, respectively, in any of these cases having the metals deposited on magnesium oxide and aluminum oxide that may result from calcination, as described above, or an LDH.

Coated Metathesis Catalysts

Representative coated metathesis catalysts comprise a metathesis inner core body and a metathesis outer shell disposed peripherally with respect to this inner core body, such as over all or at least a portion of its outer surface. The outer shell may comprise a metathesis buffering metal oxide and may substantially lack an olefin metathesis active catalytic component that is present in the inner core body. In addition to this olefin metathesis active catalytic component, the inner core body may comprise a metathesis support metal oxide, optionally with other support constituents (described below), which acts as a carrier of this component, for example with this component being dispersed uniformly or possibly non-uniformly (e.g., preferentially near the outer surface) within the metathesis support metal oxide and optional other support constituents. The metathesis support metal oxide and the metathesis buffering metal oxide may independently be selected from magnesium oxide, calcium oxide, silicon oxide (silica), strontium oxide, or other metal oxide having low acidity and therefore low cracking activity. The metathesis support metal oxide may also be aluminum oxide. For example, the metathesis support metal oxide may be magnesium oxide that is present in a mixed metal oxide, and the buffering metal oxide may be silica. As another example, the metathesis support metal oxide and the buffering metal oxide may both be silica. In some embodiments, therefore, the metathesis support metal oxide and the metathesis buffering metal oxide may be the same type of metal oxide (e.g., magnesium oxide). In more particular embodiments the primary, or possibly only, difference in composition between the metathesis inner core body and metathesis outer shell may be the presence of the olefin metathesis active catalytic component in the former and lack, or substantial lack, of this component in the latter.

According to particular embodiments relating to coated metathesis catalysts, the metathesis support metal oxide may be present in a mixed metal oxide, together with at least one further metathesis support metal oxide (as a support constituent), at various weight ratios. For example, a representative metathesis support mixed metal oxide may be an oxide of a first metal selected from the group consisting of Li, Mg, Zn, Fe, Ca, Ni, Co, Mn, and Cu, and the further metathesis support metal oxide may be an oxide of a second metal selected from the group consisting of Al, Ga, Y, In, Fe, Co, Ni, Mn, Cr, Ti, V, Zr, and La. In a specific embodiment, the metathesis inner core body may comprise a metathesis support mixed metal oxide of magnesium oxide (as the support metal oxide) and aluminum oxide (as the further metathesis support metal oxide). These may be present in the metathesis inner core body in a magnesium oxide:aluminum oxide weight ratio from about 1:1 to about 5:1 or from about 1:1 to about 3:1, such as about 3:1. Any mixed metal oxides may be present as, or derived from, a layered double hydroxide (LDH) as described above with respect to the dehydrogenation inner core body, with a magnesium-aluminum LDH being representative. Alternatively, or in combination (e.g., as a further support constituent), the metathesis inner core body may comprise aluminum oxide (alumina), silicon oxide (silica) or a mixture thereof. According to a specific example, the metathesis inner core body may comprise both a metathesis support mixed metal oxide, such as one prepared as, or derived from, an LDH (e.g., a magnesium-aluminum LDH) and further comprise another metathesis support metal oxide such as silicon oxide. According to a specific embodiment, the metathesis inner core body may comprise (i) a metathesis support mixed metal oxide that is, or that is derived from, a layered double hydroxide (LDH) and (ii) aluminum oxide (alumina), silicon oxide (silica) or a mixture thereof, with both (i) and (ii), or otherwise with only (i) or only (ii), having an olefin metathesis active catalytic component, as described herein, deposited thereon. For example, in the case of the olefin metathesis active metal being deposited onto only (ii) and not (i), the metathesis inner core body may be prepared by depositing the olefin metathesis active metal onto (ii), for example by impregnation with a solution of a precursor compound of the olefin metathesis active metal, and then physically mixing (i) with (ii) having the olefin metathesis active metal deposited thereon, preferably wherein (i) is a metathesis support mixed metal oxide that is derived from an LDH. The resulting mixture of (i) and (ii) may then be calcined as described herein. In general, therefore, either or both of the support constituents (i) and (ii) may be calcined. The support constituents (i) and/or (ii), onto which the one or more olefin metathesis active metals are dispersed, may or may not be the same constituents that are calcined. Whether or not the metathesis support metal oxide is present in, or comprises, a mixed metal oxide, this support metal oxide is preferably calcined, meaning that it has been subjected to conditions as described above with respect to the dehydrogenation support metal oxide, to improve dispersion of the olefin metathesis active catalytic component, change structural characteristics of this support metal oxide, and/or volatilize undesired impurities.

The metathesis inner core body may further comprise, as further support constituents, (i) one or more other metathesis support metal oxides and/or (ii) one or more zeolites. Representative zeolites have a structure type selected from the group consisting of FAU, FER, MEL, MTW, MWW, MOR, BEA, LTL, MFI, LTA, EMT, ERI, MAZ, MEI, and TON, and preferably selected from one or more of FAU, FER, MWW, MOR, BEA, LTL, and MFI. The structures of zeolites having these and other structure types are described, and further references are provided, in Meier, W. M, et al., Atlas of Zeolite Structure Types, 4^{th} Ed., Elsevier: Boston (1996). Specific examples include zeolite Y (FAU structure), zeolite X (FAU structure), MCM-22 (MWW structure), and ZSM-5 (MFI structure), with zeolite Y being preferred. Zeolites may be used in any of their ion (or ion exchange) forms, such as their alkali or alkaline earth metal forms (e.g., sodium form) or their hydrogen forms. In the case of zeolite Y, the hydrogen form of this zeolite (HY zeolite) is a preferred form. According to a specific embodiment, the metathesis inner core body may comprise silica and a zeolite, such as zeolite Y (e.g., HY zeolite).

In addition to a metathesis support metal oxide, the metathesis inner core body may also comprise an olefin metathesis active catalytic component, such as one or more olefin metathesis active metals (metallic elements having activity for catalyzing metathesis reactions such as reaction 2 above) dispersed on the metathesis support metal oxide as described herein. For example, an evaporative impregnation method as described above with respect to preparation of the dehydrogenation inner core body may be used, but instead with a precursor compound of an olefin metathesis active metal. Representative olefin metathesis active metals may include any one or more of those metals in Group 6 and Group 7 of the Periodic Table (e.g., tungsten). One or more of these olefin metathesis active metals may be present in the metathesis inner core body, such as dispersed uniformly or non-uniformly therein, in various oxidation states, including their zero valence or elemental metal form. A given olefin metathesis active metal may alternatively be present at other oxidation states, such as its oxide state, or otherwise in more than a single oxidation state (e.g., in a mixed oxidation state). The one or more olefin metathesis active metals, or compounds (e.g., oxides) of such metals, may be present in a combined amount generally from about 1% to about 15%, typically from about 2% to about 12%, and often from about 3% to about 10%, by weight of the metathesis inner core body.

Particular examples of metathesis inner core bodies comprise one or more of tungsten, molybdenum, and/or rhenium as olefin metathesis active metals in their oxide state (i.e., as tungsten oxide or WO₃, as molybdenum oxide or MoO₃, and/or as rhenium oxide or Re₂O₇) and further comprise a metathesis support mixed metal oxide comprising (or present as) an LDH, such as a magnesium-aluminum LDH, in which the first and second metals, M and M′, of the general LDH formula (I) above are Mg and Al, respectively.

A specific metathesis inner core body, for example, comprises tungsten oxide at a weight percent within the ranges described above with respect to compounds of olefin metathesis active metals (e.g., about 8% by weight, relative to the weight of the metathesis inner core body) deposited on a metathesis support mixed metal oxide comprising an LDH (e.g., a magnesium-aluminum LDH) and also deposited on further support constituents of the inner core body, such as another metal oxide (e.g., silica) and a zeolite (e.g., zeolite Y). The LDH may be present in an amount from generally from about 0.1 to about 80%, typically from about 0.5 to about 50%, and often from about 1 to about 30%, by weight of the metathesis inner core body. The zeolite may be present in an amount generally from about 0.1 to about 60%, typically from about 0.5 to about 30%, and often from about 1 to about 20%, by weight of the metathesis inner core body. Any other metal oxide(s) (e.g., silica) may be present in an amount representing the balance of the weight of the metathesis inner core body, such that (i) the one or more olefin metathesis active metals, or compounds (e.g., oxides) of such metals, (ii) the LDH, (iii) the other metal oxide(s), and (iv) the zeolite are present in a combined amount representing substantially all or all (e.g., greater than about 90%, greater than about 95%, or greater than about 99%) of the weight of the metathesis inner core body. The metathesis support mixed metal oxide, in addition to other metathesis support constituents, including other metal oxide(s) (e.g., silica) and/or zeolite(s) (e.g., zeolite Y), may be calcined as described above. According to a particular metathesis inner core body comprising (i), (ii), (iii), and (iv) above, the one or more olefin metathesis active metals (e.g., tungsten present as WO₃) may be dispersed on any of, or any combination of, (ii), (iii), and (iv). Also, any of, or any combination of, the support constituents (ii), (iii), and (iv) may be calcined. The support constituents (ii), (iii), and/or (iv), onto which the one or more olefin metathesis active metals are dispersed, may or may not be the same constituents that are calcined. For example, the one or more olefin metathesis active metals may be dispersed on (iii) and (iv), and not dispersed on (ii), and each of (ii), (iii), and (iv) may be calcined. In a specific example of preparing an inner core body, (iii) and (iv) may be mixed, such as in the case of preparing a physical mixture of silica and zeolite Y. The one or more olefin metathesis active metals may then be deposited onto the mixture of (iii) and (iv), for example by impregnation with a solution of a precursor compound of the olefin metathesis active metal. The resulting mixture of (iii) and (iv) (e.g., a mixture of silica and zeolite Y), having one or more olefin metathesis active metals deposited thereon, may then be calcined as described herein, optionally following drying. Alternatively, the resulting mixture of (iii) and (iv) (e.g., a mixture of silica and zeolite Y), having one or more olefin metathesis active metals deposited thereon, optionally following drying, may then be mixed with (ii), such as in the case of further mixing a mixture of silica and zeolite Y having one or more olefin metathesis active metals (e.g., tungsten) deposited thereon, with the LDH, such as a magnesium-aluminum LDH. The further resulting mixture may then be calcined to form the metathesis inner core body, for example comprising the one or more olefin metathesis active metals (e.g., tungsten present as WO₃) dispersed on a mixture of one or more metal oxides (e.g., silica) and a zeolite (e.g., zeolite Y), with the metathesis inner core body further comprising a mixed metal oxide (e.g., a magnesium-aluminum mixed metal oxide, resulting from calcination of a magnesium-aluminum LDH). According to an alternative embodiment for forming this type of metathesis inner core body, the resulting mixture of (iii) and (iv) (e.g., a mixture of silica and zeolite Y), having one or more olefin metathesis active metals deposited thereon, optionally following drying, may then be calcined. The resulting calcined, metal-impregnated material may then be combined with a mixed metal oxide (e.g., a magnesium-aluminum mixed metal oxide), resulting from (i.e., formed by) calcination of a magnesium-aluminum LDH).

Catalytic Inertness of the Outer Shell

In preferred embodiments, the dehydrogenation outer shell and/or the metathesis outer shell are substantially catalytically inert, acting beneficially as a physical separation barrier between inner cores that are catalytically active, in order to manipulate the overall contacting state of the catalyst system as described above. By being substantially catalytically inert, these outer shells exhibit little or no catalytic activity, at least in terms of catalyzing dehydrogenation or olefin metathesis, according to the catalytic activities of their respective dehydrogenation and metathesis inner cores. For example, in the case of a dehydrogenation outer shell or metathesis outer shell, this substantial lack of dehydrogenation activity or substantial lack of olefin metathesis activity may be manifested in the dehydrogenation outer shell or metathesis outer shell comprising one or more metals different from that of which the buffering metal oxide (or different from those of which the buffering mixed metal oxide and any further buffering metal oxide(s)) is the oxide, in a combined amount of generally less than about 2% by weight, typically less than about 1% by weight, and often less than about 0.2% by weight, of the outer shell. Accordingly, in certain embodiments, the dehydrogenation outer shell or metathesis outer shell may comprise any and all metals different from that of which the buffering metal oxide (or different from those of which the buffering mixed metal oxide and any further buffering metal oxide(s)) is the oxide, in these limited amounts. For example, in the case of the buffering metal oxide comprising all or substantially all magnesium oxide, the outer shell may comprise one or more metals different from magnesium, such as any and all metals different from magnesium, in these limited amounts or combined amounts, such as in the case of the outer shell comprising no metals other than magnesium. Alternatively, in the case of a dehydrogenation outer shell or metathesis outer shell, this substantial lack of dehydrogenation activity or substantial lack of olefin metathesis activity may be manifested in the dehydrogenation outer shell or metathesis outer shell comprising one or more metals not in their oxide form (e.g., in their zero valence or elemental metal form), in a combined amount of generally less than about 3% by weight, typically less than about 1% by weight, and often less than about 0.3% by weight, of the outer shell. Accordingly, in certain embodiments, the dehydrogenation outer shell or metathesis outer shell may comprise any and all metals not in their oxide form, in these limited amounts. For example, in the case of the buffering metal oxide comprising all or substantially all magnesium oxide, the outer shell may comprise one or metals in their elemental form, such as any and all metals in their elemental form, in these limited amounts, such as in the case of the outer shell comprising no metals in their elemental form.

In the case of a dehydrogenation outer shell a substantial lack of dehydrogenation activity may be manifested in the dehydrogenation outer shell comprising one or more dehydrogenation active metals of a dehydrogenation active catalytic component as described herein (e.g., one or metals selected from any of platinum, chromium, gallium, potassium, lanthanum, yttrium, ytterbium, and/or rhenium and/or one or more promoter metals selected from those in Group 13 or Group 14 of the Periodic Table) in a second combined weight percentage that is less than the combined weight percentage in which such dehydrogenation active metal(s) is/are present in the dehydrogenation inner core body. This second combined weight percentage may be made in comparison with respect to the same such dehydrogenation active metal(s) present in the dehydrogenation inner core body, or otherwise may represent that of all such dehydrogenation active metals described herein (e.g., in the case of a dehydrogenation active metal being present in the dehydrogenation outer shell but not in the dehydrogenation inner core body). In some embodiments, this second combined weight percentage may be generally less than about 2% by weight, typically less than about 0.5% by weight, and often less than about 0.1% by weight, of the dehydrogenation outer shell. In the case of a metathesis outer shell, a substantial lack of metathesis activity may be manifested in the metathesis outer shell comprising one or more olefin metathesis active metals of an olefin metathesis active catalytic component as described herein (e.g., one or metals selected from those in Group 6 or Group 7 of the Periodic Table) in a second combined weight percentage that is less than the combined weight percentage in which such olefin metathesis active metal(s) is/are present in the metathesis inner core body. This second combined weight percentage may be made in comparison with respect to the same such olefin metathesis active metal(s) present in the metathesis inner core body, or otherwise represent that of all such olefin metathesis active metals described herein (e.g., in the case of an olefin metathesis active metal being present in the metathesis outer shell but not in the metathesis inner core body). In some embodiments, this second combined weight percentage may be generally less than about 10% by weight, typically less than about 2% by weight, and often less than about 0.5% by weight, of the metathesis outer shell.

Dehydrogenation and Metathesis Catalyst Systems

The manipulation of the dehydrogenation catalyst/metathesis catalyst contacting state to obtain advantages as described herein can be achieved with catalyst systems, or at least catalyst beds (e.g., fixed beds) of such systems, in which (i) at least a portion, and possibly all, of the dehydrogenation catalyst particles in such systems, or in at least one bed of such systems, are coated, such as comprise an outer shell as described herein, (ii) at least a portion, and possibly all, of the metathesis catalyst particles in such systems are coated, such as comprise an outer shell as described herein, or (iii) both (i) and (ii). The coated portion of dehydrogenation catalyst particles may represent, for example, at least about 10% (such as from about 10% to about 99%), at least about 30% (such as from about 30% to about 95%), or at least about 50% (such as from about 50% to about 90%) of the total dehydrogenation catalyst particles in the system or at least one bed of such system. The coated portion of metathesis catalyst particles may represent, for example, at least about 15% (such as from about 15% to about 99%), at least about 20% (such as from about 20% to about 95%), or at least about 30% (such as from about 30% to about 65%) of the total metathesis catalyst particles in the system or at least one bed of such system. According to a particular embodiment, (i) at least a portion, and possibly all, of the dehydrogenation catalyst particles are not coated, such as do not comprise an outer shell as described herein (e.g., have the dehydrogenation active catalytic component dispersed uniformly, or possibly non-uniformly, throughout the dehydrogenation catalyst), and (ii) at least a portion, and possibly all, of the metathesis catalyst particles in such systems, or beds of such systems, are coated, such as comprise an outer shell as described herein. In some embodiments, (i) all or substantially all (e.g., greater than about 90%, greater than about 95%, or greater than about 99%) of the dehydrogenation catalyst particles are coated, (ii) all or substantially all (e.g., greater than about 90%, greater than about 95%, or greater than about 99%) of the metathesis catalyst particles are coated, or (iii) both (i) and (ii). According to a particular embodiment, (i) all or substantially all (e.g., greater than about 90%, greater than about 95%, or greater than about 99%) of the dehydrogenation catalyst particles are not coated (e.g., have the dehydrogenation active catalytic component dispersed uniformly, or possibly non-uniformly, throughout the dehydrogenation catalyst), and (ii) at least a portion, and possibly all (e.g., greater than about 90%, greater than about 95%, or greater than about 99%) of the metathesis catalyst particles are coated. In such particular embodiment, the coated portion of metathesis catalyst particles may represent, for example, at least about 15% (such as from about 15% to about 99%), at least about 20% (such as from about 20% to about 95%), or at least about 30% (such as from about 30% to about 65%) of the total metathesis catalyst particles in the system or at least one bed of such system. For example, a catalyst system, or at least one bed (e.g., fixed bed) of such system, may comprise a uniform mixture of (i) dehydrogenation catalyst particles, all or substantially all of which that are not coated, (ii) metathesis catalyst particles, a portion of which, including any portion within the ranges given above, are coated. Such a system or bed of a system could be, for example, a uniform mixture of (i) dehydrogenation catalyst particles that are not coated, (ii) metathesis catalyst particles that are not coated, and (iii) metathesis catalyst particles that are coated, in which (i), (ii), and (iii) are present in the system or bed at any weight ratios of (i):(ii):(iii), such as 0.1-10:0-10:0.1-10; 0.3-5:0-5:0.3-5; or 0.5-2:0-2:0.5-2. In a specific embodiment, the weight ratios of (i):(ii):(iii) are 1:1-3:1.

Representative dehydrogenation catalyst particles may comprise a dehydrogenation inner core body as described above, with coated dehydrogenation catalyst particles further comprising a dehydrogenation outer shell as described above. Representative metathesis catalyst particles may comprise a metathesis inner core body as described above, with coated metathesis catalyst particles further comprising a metathesis outer shell as described above. According to some embodiments, superior performance (e.g., in terms of conversion, selectivity, yield, and/or catalyst life) is attained with catalyst systems, or beds of such systems, in which only the metathesis catalyst particles, or portion of the metathesis catalyst particles within any of the ranges given above, further comprises a metathesis outer shell and the dehydrogenation catalyst particles do not further comprise a dehydrogenation outer shell. Superior performance in terms of catalyst life may correspond to superior catalyst stability or resistance to deactivation (e.g., through coking). Catalyst stability may be measured, for example, according to the rate of performance decrease (e.g., conversion decrease, selectivity decrease, and/or yield decrease) over the course of a given test, such as an accelerated stability test, in which constant operating conditions are maintained. Catalyst stability may alternatively be measured according to the rate of operating (e.g., catalyst bed) temperature increase, as needed to maintain a given performance (e.g., conversion, selectivity, and/or yield) over the course of a given test, such as an accelerated stability test. In some cases, superior performance in terms of combined conversion and selectivity may be attained by an increase in selectivity that overcomes (overcompensates for) a decrease in conversion, such that yield is increased. This yield increase, associated with operation at increased selectivity albeit at decreased conversion, may also be accompanied by increased catalyst life or stability, for example due to a reduced rate of formation of coke precursors that lead to catalyst coking and deactivation. Such superior performance (e.g., in terms of conversion, selectivity, yield, and/or catalyst life) may be attained relative to a comparative catalyst system, or bed of such system, that is equivalent in all respects, except that the metathesis outer shell, otherwise used to coat the metathesis inner core body of the metathesis catalyst particles or portion of these particles, is instead used, in an equivalent amount, to coat the dehydrogenation inner core body. According to other embodiments, such comparative catalyst system, or bed of such system, is equivalent in all respects, except that the metathesis outer shell is absent (i.e., both the dehydrogenation catalyst and metathesis catalysis are not coated). Regardless of whether at least a portion of the dehydrogenation catalyst particles are coated and/or at least a portion of the metathesis catalyst particles are coated, the total catalyst coating(s), such as the total or combined dehydrogenation buffering metal oxide and metathesis buffering metal oxide, may be present in the catalyst system, or bed of such system, in a combined amount generally from about 3% to about 50%, typically from about 5% to about 40%, and often from about 10% to about 35%, by weight.

According to any dehydrogenation and olefin metathesis catalyst system, or bed of such system, as described above, such system or bed may comprise (i) dehydrogenation catalyst particles comprising a dehydrogenation support metal oxide (e.g., a dehydrogenation support mixed metal oxide) and further comprising a dehydrogenation active catalytic component, and (ii) metathesis catalyst particles comprising a metathesis support metal oxide (e.g., a metathesis support mixed metal oxide) and further comprising an olefin metathesis active catalytic component. In such catalyst systems, or in at least one bed of such system, (i) at least a portion (such as all or substantially all, as described above) of the dehydrogenation catalyst particles comprise a dehydrogenation outer shell disposed peripherally about the dehydrogenation support metal oxide, the dehydrogenation outer shell comprising a dehydrogenation buffering metal oxide (e.g., substantially lacking the dehydrogenation active component, as described above) or (ii) at least a portion (such as all or substantially all, as described above) of the metathesis catalyst particles comprise a metathesis outer shell disposed peripherally about the metathesis support metal oxide, the metathesis outer shell comprising a metathesis buffering metal oxide (e.g., substantially lacking the olefin metathesis active catalytic component), or both (i) and (ii).

As described above, according to some embodiments, it may be preferable that at least a portion (such as all or substantially all, as described above) of the metathesis catalyst particles is coated and that the dehydrogenation catalyst particles are uncoated. This may be particularly desired in embodiments in which the olefin metathesis active catalytic component(s) (e.g., WO₃) is/are present in metathesis catalyst particles at a weight percentage that exceeds that at which the dehydrogenation active catalytic component(s) (e.g., Pt) is/are present in the dehydrogenation catalyst particles. In general, by coating the catalyst particles having the higher percentage of active catalytic component(s), there is a reduced tendency for the coating to block, or hinder access to, a significant proportion of these active catalytic component(s). In embodiments in which at least portions of both the dehydrogenation catalyst particles and the metathesis catalyst particles are coated, the coatings or outer shells used for these different catalysts may be of the same type or at least share a same outer shell constituent. For example, the dehydrogenation buffering metal oxide of the dehydrogenation outer shell and the metathesis buffering metal oxide of the metathesis outer shell may be of the same type, such as in the case of both being magnesium oxide or both being silicon oxide (silica). In any event, by virtue of the presence of a coating on at least a portion of at least one type of catalyst in the catalyst system, a state of contacting between catalyst types can advantageously be manipulated, such that the correct physical spacing may be achieved for desirable conversion/selectivity profiles.

In preferred embodiments, for example, the dehydrogenation catalyst particles and metathesis catalyst particles may be uniformly mixed, such as in a fixed bed of catalyst in the catalyst system, and the coating may serve to reduce the state of contacting, relative to the case of all catalyst particles being uncoated. This reduction in the state of contacting serves to beneficially reduce conversion while significantly increasing selectivity to light olefins, as described above. In the case of a mixture of dehydrogenation catalyst particles and metathesis catalyst particles of a catalyst system, or at least one bed of such system, the ratio of their respective weights may be generally from about 1:0.3-10, typically from about 1:0.5-5, and often from about 1:1-3. In representative embodiments, the dehydrogenation catalyst particles (whether coated or uncoated) and the metathesis catalyst particles (whether coated or uncoated) are present in a combined amount representing substantially all or all (e.g., greater than about 90%, greater than about 95%, or greater than about 99%) of the weight of the catalyst system, or at least one bed of such system. That is, preferably few or no types of other catalysts or other materials are present in the catalyst system, or catalyst bed of such system (e.g., catalyst bed that is disposed in a dehydrogenation/metathesis reactor).

Dehydrogenation and Metathesis Processes

Representative dehydrogenation and metathesis processes comprise contacting a feed comprising one or more paraffinic hydrocarbons such as propane with a dehydrogenation and metathesis catalyst system as described herein, to convert at least a portion of the paraffinic hydrocarbon and provide a product comprising at least one, and typically at least two, olefinic hydrocarbons having different carbon numbers, relative to the paraffinic hydrocarbon. This “paraffinic hydrocarbon” of the feed, in view of the description of two olefinic hydrocarbons having different carbon numbers, may refer to one particular hydrocarbon, such as propane, that is present in the feed at a predominant concentration as described above, possibly in a mixture with one or more other paraffinic hydrocarbons (e.g., butane) that are present at lower concentrations. In the case of the paraffinic hydrocarbon being propane, for example, two olefinic hydrocarbons present in the product, as a result of dehydrogenation and metathesis, may be ethylene and butene.

In terms of process performance, the values of conversion and selectivity described herein may, according to some embodiments, refer to the conversion of the particular paraffinic hydrocarbon that is present in the feed at the predominant concentration and to the selectivity to one or two olefinic hydrocarbons produced from dehydrogenation and metathesis of that paraffinic hydrocarbon. In other embodiments, the values of conversion and selectivity may refer to the conversion of more than one, or all, paraffinic hydrocarbons present in the feed and/or the selectivity to more than one, or all, olefinic hydrocarbons produced from dehydrogenation and metathesis of these paraffinic hydrocarbons. The values of conversion and selectivity may therefore independently and respectively refer to (i) the conversion of one or more, or all, paraffinic hydrocarbons in the feed and (ii) the selectivity to one or more, or all, olefinic hydrocarbons in the product. The conversion may be calculated, for example, by determining the weight, or weight per unit time in the case of continuous flow processes, of one or more paraffinic hydrocarbons in both the feed and the product (WPar_{feed} and WPar_{prod}), such that the conversion has a value of 1−(WPar_{prod}/WPar_{feed}), expressed as a percentage. The selectivity to one or more olefinic hydrocarbons may be calculated by determining the weight, or weight per unit time, of the one or more olefinic hydrocarbons that have been produced, i.e., weight present in the product that is absent in the feed (WO1_{prod}−WO1_{feed}), and then determining the weight, or weight per unit time, of the total hydrocarbons that have been produced (i.e., excluding the weight of hydrocarbons in the feed that remain unconverted in the product) (WHC_{prod}−WHC_{feed}, such that the selectivity has a value of (WO1_{prod}−WO1_{feed})/(WHC_{prod}−WHC_{feed}), expressed as a percentage. The yield of one or more olefinic hydrocarbons may be determined as the weight, or weight per unit time, of the one or more olefinic hydrocarbons produced, divided by the weight, or weight per unit time, of the one or more paraffinic hydrocarbons in the feed (i.e., the weight of those paraffinic hydrocarbons that could theoretically be converted to yield the one or more olefinic hydrocarbons), expressed as a percentage. For purposes of illustration using a simplified, hypothetical example, 4,400 grams (100 moles) of propane in a feed is reacted to produce, in a product, (i) 1,260 grams (45 moles) of ethylene, (ii) 2,520 grams (45 moles) of butene, and (iii) 180 grams (90 moles) of hydrogen, with 440 grams (10 moles) of propane being unconverted. Therefore, 3,780 grams (1,260+2,520) of total hydrocarbons are produced. In this case, propane conversion is 90% (1−(440/4,400)), the selectivity to ethylene is 33% ((1,260−0)/(3,780−0)), the selectivity to butene is 67% ((2,520−0)/(3,780−0)), and the selectivity to total olefins (in this case ethylene and butene combined) is 100%. The yield of ethylene is 29% (1,260/4,400), the yield of butene is 57% (2,520/4,400), and the yield of total olefins is 86% (3,780/4,400).

The performance parameters of conversion, selectivity, and/or yield may be determined on a “per-pass” or “once-through” basis, according to the total material introduced to a given dehydrogenation and metathesis catalyst system (e.g., comprising one or more catalyst beds in one or more reactors as described herein) and the total material withdrawn from the system. Often, in view of economic considerations and particularly on a larger (e.g., commercial) scale, representative processes may operate by separating and recycling unconverted paraffinic hydrocarbons. In this case, the performance parameters of conversion, selectivity, and/or yield may be determined on an “overall” basis, according to the net material introduced to the catalyst system (and excluding from the total material introduced a recycle portion that is co-introduced with the net material) and the net material withdrawn from the catalyst system (and excluding from the total material withdrawn a recycle portion that is co-withdrawn with the net material and re-introduced to the catalyst system, such as co-introduced with the net material introduced). By recycling unconverted paraffinic hydrocarbons, the overall conversion may considerably exceed the per-pass conversion. For example, as the extent of recycling increases, and approaches the condition of unconverted paraffinic hydrocarbons being recycled to extinction, the overall conversion approaches 100% (regardless of the per-pass conversion), and in this case the overall yield approaches the overall selectivity. In view of the present disclosure, those skilled in the art will appreciate the economic tradeoffs associated with increased product value due to increased yields, offset by increased costs due to increased recycle requirements. Unless stated otherwise, values of conversion, selectivity, and/or yield are given on a per-pass or once-through basis.

As discussed above, aspects of the invention relate to the manipulation of the contacting state between dehydrogenation catalysts and metathesis catalysts to provide advantageous conversion and selectivity profiles. In this regard, the per-pass conversion multiplied by the per-pass selectivity, calculated as described above, provides a per-pass yield, which directly relates to the efficiency and economic attractiveness with which a process can produce one or more olefinic hydrocarbons, such as ethylene. That is, by reducing a contacting state in order to significantly increase selectivity at the expense of only a relatively small decrease in conversion, the overall yield of the process may be augmented and its commercial viability greatly strengthened. In representative embodiments, for example, the selectivity to one or more olefinic hydrocarbons (e.g., the at least two olefinic hydrocarbons having different carbon numbers relative to the paraffinic hydrocarbon that is present in the feed at a predominant concentration) may be generally at least about 5% (e.g., from about 5% to about 90%), typically at least about 10% (e.g., from about 10% to about 85%), and often at least about 15% (e.g., from about 15% to about 80%). According to particular embodiments, the selectivity to olefinic hydrocarbons having from 2-4 carbon numbers (e.g., ethylene, propylene, and butene, in the case of a feed comprising propane) may be generally at least about 55% (e.g., from about 55% to about 99%), typically at least about 60% (e.g., from about 60% to about 96%), and often at least about 65% (e.g., from about 65% to about 94%). According to other particular embodiments, the selectivity to propylene (e.g., in the case of a feed comprising propane) may be generally at least about 20% (e.g., from about 20% to about 80%), typically at least about 25% (e.g., from about 25% to about 75%), and often at least about 30% (e.g., from about 30% to about 60%). According to yet other particular embodiments, the selectivity to ethylene (e.g., in the case of a feed comprising propane) may be generally at least about 3% (e.g., from about 3% to about 45%), typically at least about 4% (e.g., from about 4% to about 35%), and often at least about 5% (e.g., from about 5% to about 30%). According to still other particular embodiments, the selectivity to butenes (e.g., in the case of a feed comprising propane) may be generally at least about 2% (e.g., from about 2% to about 50%), typically at least about 3% (e.g., from about 3% to about 40%), and often at least about 5% (e.g., from about 5% to about 30%). In other representative embodiments, selectivities within these ranges are achieved at per-pass conversion levels of the paraffinic hydrocarbon(s) (e.g., the paraffinic hydrocarbon, such as propane, that is present in the feed in the predominant amount) of generally at least about 20% (e.g., from about 20% to about 65%), typically at least about 25% (e.g., from about 25% to about 55%), and often at least about 30% (e.g., from about 30% to about 50%). As noted above, overall conversion levels in a continuous process can be substantially increased over the per-pass conversion if unconverted paraffinic hydrocarbons are separated from the product and recycled to the feed. If essentially all of such unconverted paraffinic hydrocarbons are converted by recycling to achieve substantially 100% overall conversion (minus some inevitable losses), then the value of the overall yield will approach that of the selectivity.

In view of these per-pass conversion and selectivity values, the per-pass yield of the one or more olefinic hydrocarbons (e.g., the per-pass yield of olefinic hydrocarbons having from 2-4 carbon numbers, or otherwise the at least two olefinic hydrocarbons having different carbon numbers relative to the paraffinic hydrocarbon that is present in the feed at a predominant concentration), may be generally at least about 10% (e.g., from about 10% to about 80%), typically at least about 15% (e.g., from about 15% to about 75%), and often at least about 20% (e.g., from about 20% to about 65%). As described above, manipulating the contacting state using catalyst systems in which at least a portion of particles of one catalyst type (dehydrogenation catalyst particles or metathesis catalyst particles) are coated, can provide significant benefits in terms of selectivity and consequently yield. For example, in representative embodiments, the selectivity to, or yield of, the one or more olefinic hydrocarbons (e.g., the at least two olefinic hydrocarbons having different carbon numbers relative to the paraffinic hydrocarbon that is present in the feed at a predominant concentration) may be increased in value by a difference in percentage compared to a baseline process (i.e., the increase represented by the selectivity % of the inventive process minus the selectivity % of the baseline process or the increase represented by the yield % of the inventive process minus the yield % of the baseline process) of generally at least about 3% (e.g., from about 3% to about 35%), typically at least about 5% (e.g., from about 5% to about 30%), and often at least about 7% (e.g., from about 7% to about 15%). For example, the increases in these ranges over a baseline process may represent increases in selectivity to olefinic hydrocarbons having from 2-4 carbon numbers, or otherwise increases in selectivity to propylene, ethylene, or butenes. In such embodiments, the baseline process is similar in all respects (catalysts, feed composition, reactor configuration, process conditions, etc.), with the exception that none of the dehydrogenation catalyst particles is coated (e.g., comprises an outer shell as described herein) and none of the metathesis catalyst particles is coated (e.g., comprises an outer shell as described herein).

Contacting between the feed and catalyst system can be performed in a batchwise (discontinuous) manner, but, for the sake of process efficiency, normally a continuous flow of feed is input to the catalyst system, such as in the form of one or more fixed beds or possibly a fluidized bed that is contained within a dehydrogenation/metathesis reactor, and a continuous flow of product is withdrawn from the catalyst system. In the case of a catalyst system having two or more fixed beds, any two or more of such beds may be contained within a single dehydrogenation/metathesis reactor, for example divided using a layer of inert material or otherwise stacked directly adjacent one another, in order to further regulate the contacting state along a spectrum of contacting states as described above. Alternatively, any two or more of such beds may be contained within separate dehydrogenation reactors, dehydrogenation/metathesis reactors, or metathesis reactors, for example if separate reaction conditions (e.g., temperature, pressure, and/or weight hourly space velocity) are desired for such beds. Therefore, various configurations are possible in the case of a catalyst system having three fixed beds, such as first, second and third beds, with the first bed being disposed upstream of the second bed and the third bed being disposed downstream of the second bed, with the relative positional terms “upstream” and “downstream” being with respect to a direction of flow of the feed. For example (i) all three beds may be contained within a single reactor, (ii) the first and second beds may be contained in an upstream reactor and the third bed in a downstream reactor, (iii) the first bed may be contained in an upstream reactor and the second and third beds may be contained in a downstream reactor, or (iv) each of the three beds may be contained in a separate reactor. In view of this description, the possibilities for configurations of catalyst systems having four or more beds, although more numerous, are nonetheless apparent.

In the case of a catalyst system having two or more fixed beds, individual beds may comprise (i) all or substantially all (e.g., greater than about 90% by weight, greater than about 95% by weight, or greater than about 99% by weight) of dehydrogenation catalyst particles, (ii) all or substantially all (e.g., greater than about 90% by weight, greater than about 95% by weight, or greater than about 99% by weight) of metathesis catalyst particles, or (iii) a mixture of dehydrogenation catalyst particles and metathesis catalyst particles, such as in a ratio of their respective weights as described above. In any of such individual beds of a given system, all or a portion of dehydrogenation catalyst particles, or all or a portion of the metathesis catalyst particles, may be coated, such as portions within the ranges described above with respect to the coated portions of the dehydrogenation catalyst or metathesis catalyst. For example, a catalyst system may comprise three catalyst beds comprising, (i) a first or upstream bed comprising all dehydrogenation catalyst particles that are not coated, (ii) a second or middle bed comprising a mixture of dehydrogenation catalyst particles that are not coated, metathesis catalyst particles that are not coated, and metathesis catalyst particles that are coated, and (iii) a third or downstream bed comprising all metathesis catalyst particles that are not coated. In view of the present disclosure, it can be appreciated that numerous possibilities exist, with respect to individual beds of a catalyst system, their upstream or downstream position relative to one another, their containment in separate reactors or in combination in a single reactor, their content of dehydrogenation catalyst and/or metathesis catalyst, and the portions of these catalysts that are coated.

In the case of a continuous flow system, the feed, comprising paraffinic hydrocarbon(s) as described above, can refer to a single feed stream or a total combined feed stream that includes, for example, other sources of paraffinic hydrocarbon(s) such as a recycle stream as described above that comprises an unconverted portion of the paraffinic hydrocarbon(s) that has been separated from the product. The hydrocarbon feed may, but does not necessarily, comprise only paraffinic hydrocarbons. For example, the feed generally comprises predominantly (i.e., at least 50% by weight) paraffinic hydrocarbons, typically comprises at least about 80% (e.g., from about 80% to about 100%) paraffinic hydrocarbons, and often comprises at least about 90% (e.g., from about 90% to about 100% by weight) paraffinic hydrocarbons. Any of these ranges may refer to a combined amount of paraffinic hydrocarbons present in the feed, or these ranges may alternatively refer to a particular paraffinic hydrocarbon, such as propane or butane. Light paraffinic hydrocarbons, e.g., C₂-C₆ paraffins, that may be present in a feed as described herein, may be obtained as products or fractions from crude oil refining, such as light gas oil, including liquefied petroleum gas (LPG) and naphtha. Of the overall weight of hydrocarbons present in the feed, generally at least 50% by weight, such as from about 60% to about 100% by weight, are paraffinic hydrocarbons. In many cases all or a large proportion, such as from about 80% to about 100% or even from about 90% to about 100%, of the paraffinic hydrocarbons in the feed are a paraffinic hydrocarbon having a particular carbon number, such as a carbon number of 3 or 4 (in the case of propane or butane, respectively).

Representative dehydrogenation and metathesis processes, for example to achieve the conversion, selectivity, and/or yield values as described herein, therefore comprise contacting a feed, either continuously or batchwise, with a catalyst system as described herein, in which at least a portion of one or both of the dehydrogenation or metathesis catalysts are coated. Generally, the contacting is performed with the feed being passed continuously through a catalyst system as described above, and preferably having at least one (and possibly only one) fixed bed of a mixture of the catalysts, and preferably a uniform mixture, in a reactor or reaction zone, normally under conditions effective for achieving the desired reaction sequence. In some cases, a swing bed system may be utilized, in which the flowing paraffinic hydrocarbon-containing feed is periodically re-routed to (i) bypass one or more beds of catalyst that have become spent or deactivated (spent catalyst system) and (ii) contact one or more beds of fresh catalyst (fresh catalyst system). A number of other suitable configurations for carrying out the feed/catalyst contacting are known in the art, with the optimal choice depending on the particular feed, rate of catalyst deactivation, and other factors. Such configurations include moving bed configurations (e.g., counter-current flow configurations, radial flow configurations, etc.) and fluidized bed configurations, any of which may be integrated with batchwise or continuous catalyst regeneration, as is known in the art.

Representative conditions for contacting of the feed with the catalyst system, at which the above per-pass conversion, selectivity, and yield may be obtained, include a temperature generally from about 350° C. to about 800° C., typically from about 500° C. to about 650° C. and often from about 550° C. to about 625° C.; an absolute pressure generally from about 0.1 bar to about 100 bar, typically from about 1 bar to about 50 bar, and often from about 1 bar to about 25 bar; and a weight hourly space velocity (WHSV) generally from about 0.01 hr⁻¹ to about 20 hr⁻¹, typically from about 0.01 hr⁻¹ to about 10 hr⁻¹, and often from about 0.1 hr⁻¹ to about 5 hr⁻¹. As is understood in the art, the WHSV is the weight flow of the feed divided by the weight of the catalyst (e.g., present in one or more beds as described above) and represents the equivalent catalyst weights of feed processed every hour. The WHSV is related to the inverse of the reactor residence time. Under the reaction conditions, the feed is normally partially or all in the vapor phase in the reactor or reaction zone containing the catalyst system, but it may also be in the liquid phase, depending on the particular process conditions and feed used.

The following examples are set forth as representative of the present invention. These examples are not to be construed as limiting the scope of the invention as other equivalent embodiments will be apparent in view of the present disclosure and appended claims.

### EXAMPLES

Experiments were conducted to evaluate catalyst systems for performing (i) the dehydrogenation of propane to propylene and (ii) the metathesis of propylene to ethylene and butene. This integration of reactions was also termed “propane transformation.” Each catalyst system tested included both a dehydrogenation catalyst and either one type of metathesis catalyst or two types of metathesis catalysts. In some examples, catalysts were mixed uniformly and in other examples catalysts were disposed in separate beds, such as separated by a distance. In the case of the examples termed “comparative,” neither the dehydrogenation catalyst nor the metathesis catalyst was a coated catalyst. In the case of examples termed “inventive,” one type of metathesis catalyst was coated with an outer shell of buffering silica, disposed peripherally about an inner core body having tungsten as an olefin metathesis active metal. For the catalyst systems evaluated, the designations used for the dehydrogenation catalysts and metathesis catalysts and their corresponding compositions are summarized in Table 1.

TABLE 1
Catalyst Designations

| Catalyst Type | Designation | Description/Composition |
|---|---|---|
| dehydrogenation | DEHY A | 0.3 wt-% Pt/0.6 wt-% Sn, supported on mixed |
| | | MgO—Al₂O₃ that was obtained from calcination of an LDH |
| dehydrogenation | DEHY B | 0.3 wt-% Pt/0.6 wt-% In, supported on mixed |
| | | MgO—Al₂O₃ that was obtained from calcination of an LDH |
| metathesis | META A | 8 wt-% tungsten, as WO₃, supported on a mixture of 5 |
| | | wt-% hydrogen form zeolite Y (HY zeolite), 10 wt-% |
| | | mixed MgO—Al₂O₃ that was obtained from calcination of |
| | | an LDH, with the balance of the catalyst weight being |
| | | SiO₂ |
| metathesis | META B | 8 wt-% tungsten, as WO₃, supported on a mixture of 5 |
| | | wt-% hydrogen form zeolite Y (HY zeolite), with the |
| | | balance of the catalyst weight being SiO₂. The support |
| | | component of the mixed MgO—Al₂O₃, present in catalyst |
| | | META A, was therefore lacking in catalyst META B |
| metathesis | COATED | metathesis inner core body having the composition of |
| | META A | META A, but coated with SiO₂ |
| metathesis | COATED | metathesis inner core body having the composition of |
| | META B | META B, but coated with SiO₂ |

Preparation of Dehydrogenation Catalysts and Metathesis Catalysts

The catalyst designated as DEHY A was prepared as follows: An Mg—Al—CO₃ layered double hydroxide was calcined in air at 620° C. (1148° F.) for 8 hours to obtain a mixed magnesium oxide (MgO)-aluminum oxide (Al₂O₃) support. This calcined LDH, or mixed metal oxide, was impregnated with Pt and Sn metals using impregnation solutions of chloroplatinic acid and tin (II) chloride dihydrate. Following the combining of the mixed metal oxide and impregnation solutions, the wet particles obtained were dried at 120° C. (248° F.) for 12 hours and calcined under air at 620° C. (1148° F.) for 2 hours. The resulting non-coated dehydrogenation catalyst contained 0.3 wt-% Pt and 0.6 wt-% Sn, on the MgO—Al₂O₃ support, with these weight percentages being based on the total catalyst weight. The catalyst designated DEHY B was prepared in a similar manner, except that the tin (II) chloride dihydrate impregnation solution was replaced with an indium nitrate hydrate impregnation solution. The resulting non-coated dehydrogenation catalyst contained 0.3 wt-% Pt and 0.6 wt-% In, on the MgO—Al₂O₃ support, with these weight percentages being based on the total catalyst weight.

The catalyst designated as META A was prepared as follows: A support was prepared by mixing SiO₂ with hydrogen form of zeolite Y (HY-Zeolite), and particles of this mixture of solids were impregnated using an impregnation solution of ammonium metatungstate hydrate. Following the combining of these particles and impregnation solution, the wet, impregnated particles obtained were dried at 120° C. (248° F.) for 12 hours and calcined under air at 550° C. (1022° F.) for 8 hours. In addition, an Mg—Al—CO₃ LDH was calcined under air at 620° C. (1148° F.) for 8 hours to convert the LDH to an MgO-Al₂O₃ mixed metal oxide (calcined LDH). The calcined, tungsten-impregnated material and MgO—Al₂O₃ mixed metal oxide material were combined to obtain a non-coated metathesis catalyst, which contained, based on the total catalyst weight, 8 wt-% W, 5 wt-% HY-zeolite, and 10 wt-% mixed magnesium oxide (MgO)-aluminum oxide (Al₂O₃), with the balance being SiO₂. The catalyst designated META B, was prepared in a similar manner, except that the step of mixing with the Mg—Al—CO₃ layered double hydroxide was omitted and the proportions of the remaining ingredients adjusted, such that the resulting non-coated metathesis catalyst contained, based on the total catalyst weight, 8 wt-% W, and 5 wt-% HY-zeolite, with the balance being SiO₂.

The catalysts designated as COATED META A and COATED META B were prepared as coated catalysts, using particles of the catalysts designated as META A and META B, respectively, as inner core bodies. In the coated catalysts, these inner core bodies were coated with mesoporous silica (mSiO₂), using tetraethoxysilane (TEOS) as the source of this silica, cetyltrimethylammonium bromide (CTAB) as a templating surfactant, and an ethanol-water solution as a solvent. To prepare a coating solution, 170 grams of water was mixed with 90 grams of ethanol and 3.5 grams of CTAB. Then, 3 milliliters of aqueous ammonium hydroxide solution (˜30% NH₄OH) was added to this mixture, which was left stirring for 30 minutes. Following this period, 20 grams of TEOS were introduced slowly (dropwise) while maintaining the stirring. Approximately 5.6 grams of SiO₂ as a coating solution, made by this procedure, was used to coat, in each case, (i) particles of the catalyst META A, in the formation of the catalyst designated as COATED META A, and (ii) particles of the catalyst META B, in the formation of the catalyst designated as COATED META B. In each case, coating of the inner core bodies, corresponding respectively to the catalysts designated as META A and META B, involved adding the uncoated catalyst particles into the coating solution (thereby performing immersion or dip coating) and stirring for an additional 24 hours. This was followed by filtering and rinsing the filtered particles several times with deionized water, and allowing the wet particles to partially dry at room temperature for at least 1 hour. The resulting, partially dry solid mixture was then more completely dried at 120° C. (248° F.) for 12 hours and calcined under air at 550° C. (1022° F.) for 8 hours, to yield the coated catalysts.

As an alternative to immersion or dip coating, the coating solution as described above can be spray coated onto the catalyst inner core bodies, for example using a spray coating machine to provide homogeneous, coated catalyst particles with a substantially uniform amount of coating material, as an outermost layer, on each particle. Following such spray coating, the same room temperature and elevated temperature drying steps, as well as the same calcination step, can be applied as described above, to yield the coated catalysts.

A transmission electron microscopy (TEM) image of a metathesis catalyst particle, prepared as described above with respect to the catalyst designated as META A, is shown in FIG. 1A, whereas a TEM image of a metathesis catalyst particle coated with mesoporous silica (mSiO₂) and prepared as described above with respect to the catalyst designated as COATED META A, is shown in FIG. 1B. The images, showing features on the nanoscale, are relatively darker in those areas that are thick and dense and therefore more opaque with respect to passage of the imaging electron beam, and conversely relatively lighter in thin areas that allow greater passage of this beam. FIG. 1A, showing more specifically the edge of a non-coated metathesis catalyst particle, illustrates that this edge is well defined and smooth. In contrast, the edge of the coated metathesis catalyst particle shown in FIG. 1B illustrates crystal growth associated with the mSiO₂. The coating at the outer edge of the catalyst particle therefore serves to form a barrier against direct contact of its catalytically active, inner core, with adjacent catalyst particles. In this manner, a contacting state of the catalyst system can be effectively regulated to achieve a desired conversion/selectivity profile as described herein. FIG. 2, which is an image of the same catalyst sample as shown in FIG. 1B, shows the porous structure throughout the coated metathesis catalyst, as evidenced by the numerous patches of light and dark areas. These areas indicate a layered (laminated) structure of the mSiO₂, disposed peripherally about the inner core body. Additionally, the black spots, which are more easily visible in the image of FIG. 2, correspond to tungsten nanoparticles. This was separately confirmed by analysis using Transmission Electron Microscopy Energy-Dispersive X-ray Spectroscopy (TEM-EDX).

Catalyst Systems Using the Prepared Catalysts

The catalyst systems evaluated, which each included one of the dehydrogenation catalysts and either one or two of the metathesis catalysts described in Table 1, are summarized in Table 2 below. The weight ratios are given in relative weights of [dehydrogenation catalyst]:[first metathesis catalyst]:[second metathesis catalyst, if used]. As indicated in this table, three comparative examples were performed, using catalyst systems without a coated catalyst, and two inventive examples were performed, using catalyst systems having the coated metathesis catalysts, designated COATED META A (in Inventive Example #1) and COATED META B (in Inventive Example #2).

TABLE 2
Catalyst Systems Evaluated

| | Dehydro- | First | Second | | |
|---|---|---|---|---|---|
| | genation | Metathesis | Metathesis | Weight | |
| Example | Catalyst | Catalyst | Catalyst | Ratio | System Type |
| Comparative | DEHY A | META A | | 1:2.67 | Separate bed of META A, |
| Example #1 | | | | | downstream of bed of |
| | | | | | DEHY A, with no |
| | | | | | separation (0 cm) |
| Comparative | DEHY A | META A | | 1:2.67 | Separate bed of META A, |
| Example #2 | | | | | downstream of bed of |
| | | | | | DEHY A, with 2 cm |
| | | | | | separation |
| Comparative | DEHY A | META A | | 1:2.67 | Single bed of uniform |
| Example #3 | | | | | mixture of DEHY A and |
| | | | | | META A |
| Inventive | DEHY A | META A | COATED | 1:1.67:1 | Single bed of uniform |
| Example #1 | | | META A | | mixture of DEHY A, |
| | | | | | META A, and COATED |
| | | | | | META A |
| Inventive | DEHY B | META B | COATED | 1:1.67:1 | Single bed of uniform |
| Example #2 | | | META B | | mixture of DEHY A, |
| | | | | | META B, and COATED |
| | | | | | META B |

Testing of the Catalyst Systems for Integrated Propane Dehydrogenation/Metathesis

For testing of the catalyst systems described above in Table 2, each system was initially treated by heating to 580° C. (1076° F.) and maintaining this temperature under flowing O₂ for 30 minutes and then under flowing H₂ for 30 minutes. Thereafter each catalyst system was allowed to cool to an operating temperature of 570° C. (1058° F.), and propane flow was then initiated to obtain a WHSV of about 1.24 hr in each case, with an operating pressure of 1 bar gauge (about 2 bar absolute). With respect to Comparative Example #1 and Comparative Example #2, and consistent with the System Type as shown above in Table 2, the flowing propane feed was first contacted with the upstream bed of the catalyst designated as DEHY A and the effluent from this bed then contacted with the downstream bed of the catalyst designated META A. Whereas in Comparative Example #1 these beds were directly adjacent, in Comparative Example #2 these beds were separated with an inert material by an intervening distance of 2 cm, thereby lowering the contacting state between these catalyst types. In each of these comparative examples, the weight ratio of DEHY A to META A was 1:2.67. Also consistent with the System Type as shown above in Table 2, (i) Comparative Example #3 evaluated a catalyst system of a single bed of a uniform mixture of the catalysts designated DEHY A and META A, at a weight ratio of 1:2.67; (ii) Inventive Example #1 evaluated a catalyst system of a single bed of a uniform mixture of the catalysts designated DEHY A, META A, and COATED META A, at a weight ratio of 1:1.67:1; and (iii) Inventive Example #2 evaluated a catalyst system of a single bed of a uniform mixture of the catalysts designated DEHY B, META B, and COATED META B, at a weight ratio of 1:1.67:1.

In the evaluation of each catalyst system, samples of the effluent from that system (i.e., exiting the downstream bed of the catalyst designated META A in Comparative Example #1 and Comparative Example #2, or exiting the single bed in Comparative Example #3, Inventive Example #1, and Inventive Example #2) were obtained after 3 hours on stream and 6 hours on stream. These samples were quantitatively analyzed for their composition (by gas chromatography) in order to determine propane conversion and selectivity to all olefins (including olefins having 5 carbon atoms or greater, C₅⁺), as well as the selectivities to the individual compounds, propylene and ethylene, and selectivity to butene isomers (C₄ olefins). The yield of all olefins, as well as the yields of propylene, ethylene, and butene isomers, can be determined as the product of the propane conversion and the respective selectivities in each case (e.g., the yield of all olefins as the product of the propane conversion and selectivity to all olefins, the yield of propylene as the product of propane conversion and selectivity to propylene, the yield of ethylene as the product of propane conversion and selectivity to ethylene, or the yield of butene isomers as the product of propane conversion and selectivity to butene isomers). Also determined from the sample analysis, but not shown in Table 3 below, were the selectivities to all paraffins, as well as the selectivities to the individual compounds, methane and ethane. The following conversion and selectivity results are provided in Table 3.

TABLE 3
Calculated Conversion and Product Selectivities

| | % | | % | | % | | % | | % | |
|---|---|---|---|---|---|---|---|---|---|---|
| | Propane | | Selectivity, | | Selectivity, | | Selectivity, | | Selectivity, | |
| | Conversion | | Total Olefins | | Propylene | | Ethylene | | Butenes | |
| Example | 3 hr | 6 hr | 3 hr | 6 hr | 3 hr | 6 hr | 3 hr | 6 hr | 3 hr | 6 hr |
| Comparative | 37.5 | 37.6 | 85.2 | 83.9 | 42.4 | 38.3 | 14.2 | 15.1 | 20.8 | 20.3 |
| Example #1 | | | | | | | | | | |
| Comparative | 36.9 | 36.7 | 86.9 | 85.3 | 50.0 | 40.0 | 11.8 | 14.9 | 19.6 | 20.8 |
| Example #2 | | | | | | | | | | |
| Comparative | 41.4 | 33.8 | 74.3 | 77.1 | 53.0 | 47.6 | 2.57 | 6.85 | 12.9 | 16.0 |
| Example #3 | | | | | | | | | | |
| Inventive | 40.6 | 36.2 | 83.8 | 84.1 | 45.6 | 38.9 | 11.6 | 14.9 | 20.4 | 20.2 |
| Example #1 | | | | | | | | | | |
| Inventive | 39.1 | 37.0 | 82.2 | 82.9 | 41.9 | 39.4 | 14.0 | 15.7 | 18.9 | 18.8 |
| Example #2 | | | | | | | | | | |

From the results above, it can be seen that the catalyst system tested in Comparative Example #3 provided higher propane conversion relative to that obtained in Comparative Examples #1 and #2, but lower selectivity to (and also a lower yield of) total olefins, and particularly ethylene and butenes. Based on the sample analyses, it could be determined that the selectivity loss resulted from significantly greater conversion to undesired paraffins, and particularly ethane, in Comparative Example #3, relative to Comparative Examples #1 and #2. However, it can be seen that selectivity to (and also the yield of) total olefins can be increased, without a loss in propane conversion, using the catalyst systems tested in Inventive Examples #1 and #2. As demonstrated in these examples, by having at least a fraction of the metathesis catalyst present in the system as coated catalyst in order to regulate the contacting state, conversion can be maintained, while selectivity to olefins, and particularly ethylene and butene isomers, can be significantly increased. Moreover, the data above show that the catalyst systems tested in Inventive Examples #1 and #2 exhibited superior stability, on the basis of the calculated conversion and selectivity values at 3 and 6 hours on stream, compared to the catalyst system tested in Comparative Example #3.

Overall aspects of the invention are directed to processes that exploit unexpected findings relating to the state of contacting between dehydrogenation and metathesis catalysts and more particularly its impact on the conversion and selectivity profiles achieved in combined dehydrogenation and metathesis processes. In particular, this contacting state can be advantageously manipulated through the application of a coating to one or both catalyst types and thereby maintain a degree of physical separation. Those having skill in the art, with the knowledge gained from the present disclosure, will recognize that various changes can be made in the above catalysts, catalyst systems, and processes using these catalyst systems, without departing from the scope of the present disclosure.

1. A coated dehydrogenation catalyst comprising:
a dehydrogenation inner core body comprising a dehydrogenation support metal oxide and further comprising a dehydrogenation active catalytic component, and
a dehydrogenation outer shell disposed peripherally about the dehydrogenation inner core body, the dehydrogenation outer shell comprising a dehydrogenation buffering metal oxide.

2. The coated dehydrogenation catalyst of claim 1, wherein the dehydrogenation support metal oxide and the dehydrogenation buffering metal oxide are of the same type.

3. The coated dehydrogenation catalyst of claim 2, wherein the dehydrogenation support metal oxide and the buffering metal oxide are both magnesium oxide.

4. The coated dehydrogenation catalyst of claim 1, wherein the dehydrogenation inner core body comprises a dehydrogenation support mixed metal oxide of the dehydrogenation support metal oxide and at least one further dehydrogenation support metal oxide.

5. The coated dehydrogenation catalyst of claim 4, wherein the dehydrogenation support metal oxide is an oxide of a first metal selected from the group consisting of Li, Mg, Zn, Fe, Ca, Ni, Co, Mn, and Cu, and wherein the further dehydrogenation support metal oxide is an oxide of a second metal selected from the group consisting of Al, Ga, Y, In, Fe, Co, Ni, Mn, Cr, Ti, V, Zr, and La.

6. The coated dehydrogenation catalyst of claim 5, wherein the first metal is magnesium and the second metal is aluminum.

7. The coated dehydrogenation catalyst of claim 6, wherein the magnesium oxide and aluminum oxide are present in the dehydrogenation inner core body in a magnesium oxide:aluminum oxide weight ratio from about 1:1 to about 5:1.

8-20. (canceled)

21. A coated metathesis catalyst comprising:
a metathesis inner core body comprising a metathesis support metal oxide and further comprising an olefin metathesis active catalytic component, and
a metathesis outer shell disposed peripherally about the metathesis inner core body, the metathesis outer shell comprising a metathesis buffering metal oxide.

22. The coated metathesis catalyst of claim 21, wherein the olefin metathesis active catalytic component comprises one or more olefin metathesis active metals selected from the group consisting of metals in Group 6 and Group 7 of the Periodic Table.

23. The coated metathesis catalyst of claim 21, wherein the metathesis support metal oxide is silicon oxide (silica) or aluminum oxide.

24. The coated metathesis catalyst of claim 21, wherein the metathesis buffering metal oxide is selected from the group consisting of magnesium oxide, calcium oxide, silicon oxide (silica), and strontium oxide

25. The coated metathesis catalyst of claim 21, wherein the metathesis inner core body further comprises a zeolite.

26. The coated metathesis catalyst of claim 21, wherein the metathesis support metal oxide is present in a support mixed metal oxide, together with at least one further metathesis support metal oxide.

27. The coated metathesis catalyst of claim 27, wherein the support mixed metal oxide is an oxide of a first metal selected from the group consisting of Li, Mg, Zn, Fe, Ca, Ni, Co, Mn, and Cu, and the further metathesis support metal oxide is an oxide of a second metal selected from the group consisting of Al, Ga, Y, In, Fe, Co, Ni, Mn, Cr, Ti, V, Zr, and La.

28-30. (canceled)

31. A dehydrogenation and metathesis catalyst system comprising:
dehydrogenation catalyst particles comprising a dehydrogenation support metal oxide and further comprising a dehydrogenation active catalytic component, and
metathesis catalyst particles comprising a metathesis support metal oxide and further comprising an olefin metathesis active catalytic component, wherein
(i) at least a portion of the dehydrogenation catalyst particles further comprise a dehydrogenation outer shell disposed peripherally about the dehydrogenation support metal oxide, the dehydrogenation outer shell comprising a dehydrogenation buffering metal oxide, or
(ii) at least a portion of the olefin metathesis catalyst particles further comprise an olefin metathesis outer shell disposed peripherally about the olefin metathesis support metal oxide, the olefin metathesis outer shell comprising an olefin metathesis buffering metal oxide,
or both (i) and (ii).

32. The dehydrogenation and metathesis catalyst system of claim 31, wherein a weight ratio of dehydrogenation catalyst particles:olefin metathesis catalyst particles is from about 1:1-3.

33. The dehydrogenation and metathesis catalyst system of claim 31, wherein the dehydrogenation buffering metal oxide and the olefin metathesis buffering metal oxide are of the same type.

34. (canceled)

35. A dehydrogenation and metathesis process, the process comprising contacting a feed comprising a paraffinic hydrocarbon with the dehydrogenation and olefin metathesis catalyst system of claim 31 to convert at least a portion of the paraffinic hydrocarbon and provide a product comprising at least one olefinic hydrocarbon having a different carbon number, relative to the paraffinic hydrocarbon.

36. The process of claim 35, wherein the paraffinic hydrocarbon is propane and the at least two olefinic hydrocarbons are ethylene and butene.

37. The process of claim 35, wherein the feed is in a gas phase and the paraffinic hydrocarbon is present in the feed in an amount of at least about 80% by volume.

38-42. (canceled)
